(19) United States
(12) Patent Application Publication
(10) Pub. No.: US 2009/0120809 A1
(43) Pub. Date: May 14, 2009

(54) Nitroreductase biosensors for detecting nitro-compounds

(75) Inventors: Maher Kalaji, Anglesey (GB); Peter Anthony Williams, Anglesey (GB); Christopher David Gwenin, Isle of Anglesey (GB)

Correspondence Address: VENABLE LLP, P.O. BOX 34385, WASHINGTON, DC, 20043-9998, US

(73) Assignees: UNIVERSITY OF WALES, BANGOR, Gwynned, WL (GB); TRWYN LIMITED, Anglesley, WL (GB)

(21) Appl. No.: 10/582,557

(22) PCT Filed: Nov. 17, 2004
(86) PCT No.: PCT/GB04/04817

§ 371 Date: Jun. 4, 2007

(30) Foreign Application Priority Data
Dec. 11, 2003 (GB) 0328784.4

(51) Int. Cl. G01N 27/327; C12N 9/06; C07H 21/00

(52) U.S. Cl. 205/780.5; 204/403.01; 435/191; 536/23.2

(57) ABSTRACT

This invention provides a sensing device comprising an electrode comprising a noble metal layer, on which layer is located a biological material having nitroreductase activity. This invention further provides a method of detecting nitro group containing compounds, the method comprising the steps of:
(a) providing a sensing device of the first aspect of the invention and a reference electrode;
(b) applying a potential between the electrodes;
(c) measuring the current;
(d) contacting the sensing device with a sample of substrate material to be tested; and
(e) measuring the current change.

### FIELD OF THE INVENTION

This invention relates to biosensors, methods of sensing nitro-compounds and modified enzymes per se. In particular, but not exclusively the invention relates to biosensors, methods of detecting nitro-compounds and modified enzymes useful in detecting explosives.

### BACKGROUND TO THE INVENTION

Biosensors generally comprise a class of devices that recognise a desired compound (analyte) in a sample and generate a signal which can be resolved to determine the concentration of the compound within the sample. Most biosensors are based on their ability to distinguish a specific analyte, or limited range of analytes, without the need for separation or isolation. For example there are known biosensors which can detect the presence of particular compounds within a blood or water sample directly, thereby eliminating the need for a lengthy or complex purification steps to recover the analyte of interest.

Many biosensors rely on the coupling of a recognition system with an electrochemical or optical transducer to produce an electrical or optical signal or impulse when the recognition system recognises an analyte, which is then used in analyte concentration determination. Electrochemical transducers used in biosensors include potentiometric and amperometric transducer mechanisms. Optical based transducer mechanisms include fluorescence, phosphorescence and a simple colour change.

In potentiometric-based biosensors the accumulation of charge density at the surface of an electrode is measured and is representative of the concentration of analytes to which a biosensor is exposed. In amperometric sensors, electrons that are exchanged between a biological system and an electrode generate a current which may be monitored to determine the concentration of analytes within their sample. Amperometric sensors are commonly employed in blood glucose and ethanol sensors, as well as other devices which monitor compounds of biological significance. In many biosensors a biological recognition molecule performs part of the sensor. The biological recognition molecule may be a nucleic acid sequence, an RNA, or more commonly a protein such as an enzyme or antibody. The biological recognition molecule binds specific analytes, or a limited range of analytes, and is therefore ideally suited for selective detection of specific analytes.

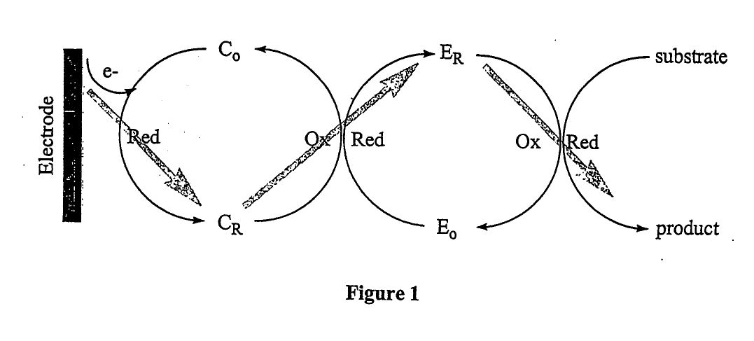

Charge transfer is either accomplished by low molecular weight redox co-factors, such as the NAD⁺/NADH or by the direct interaction of the redox centres of proteins. Both types of biochemical charge transfer reactions have been previously coupled to redox electrodes. Electrochemistry provides a useful tool for studying the redox chemistry associated with enzymes. A method that can be used to evaluate electron transfer between enzymes and an electrode is the indirect method of using a small redox molecule serving as an electron transfer co-factor. The scheme of the electron transfer coupling is illustrated in FIG. 1, where the enzymatic (E_{O}/E_{R}) reaction for the oxidation (or reduction) of the substrate is linked to the electrochemical reduction (or oxidation) of the co-factor (C_{O}/C_{R}) by the electrode as a final electron acceptor (or donor). The enzyme catalysed electrochemical oxidation (or reduction) of the substrate is called bioelectrocatalysis.

The enzyme electrode is a combination of any electrochemical probe (amperometric, potentiometric or conductometric) with a thin layer (10-200 μm) of immobilised enzyme. In these devices, the function of the enzyme is to provide selectivity by virtue of its biological affinity for a particular substrate molecule.

However there are problems in attaching many biological recognition molecules to biosensors. Many biological recognition molecules such as enzymes, and antibodies have “active sites” which must be presented to the analyte in order for the analyte to bind the biological recognition molecule. For many biological recognition molecules, attachment to a biosensor may obscure or hinder the active sites, and therefore render the biological recognition molecule less effective, or, in some cases, inactive.

Of growing interest is the detection of nitro compounds present in many explosives and explosive precursors such as fertiliser.

Interest is growing not only for the detection of buried munitions from previous wars, but also through interest in detecting explosives on a person's body, a person's possessions, vehicles and structures.

For buried or unexploded munitions, there is a great hazard that members of the public working in areas where the explosives are buried, may plough up or step on unexploded ordinance, without being aware that explosives are present at all.

Currently there are sensing systems available to detect explosives, which include for instance the system described in U.S. Pat. No. 5,972,638, in which a modified organism of the Pseudomonas species or Bacillus species are sprayed onto ground believed to contain buried explosives. The ground is gently irradiated before spraying on the organism, in order to increase explosive vapour concentration in the soil. This system is relatively expensive, and requires equipment in the form of crop spraying aircraft, spraying devices and irradiation devices. Furthermore, the ground requires substantial quantities of modified organism to be sprayed onto the surface, in order that a detectable signal is achievable.

In WO 97/03201 and GB 2303136, nitroreductase enzymes per se are purified and used to detect nitrates in samples of soil or otherwise, by way of a calorimetric method. The nitroreductase used in these patents is a pentaerythritol tetra nitratereductase, which is specific only for PETN. Furthermore, the concentration level of nitrates which can be detected is relatively high and in order for detection to take place, a soil sample must be removed from the ground, and taken away for a relatively time consuming and complicated assay procedure.

It would therefore be advantageous to provide a biosensor, able to detect explosives in situ, whether in soil or other ground material, or in a sample of material.

It would furthermore be advantageous to provide a highly sensitive detection system for nitro-compounds commonly present in explosives and fertilizers, which is able to detect in at least the nanomolar, but more preferably the picomolar concentration range.

It is an aim of preferred embodiments of the present invention to overcome or mitigate at least one problem of the prior art, whether expressly disclosed herein or not.

### SUMMARY OF THE INVENTION

According to a first aspect of the invention there is provided a sensing device comprising an electrode comprising a noble metal layer, on which layer is located a biological material having nitroreductase activity.

Preferably the noble metal layer comprises a noble metal selected from the group consisting of gold, silver, platinum, palladium, iridium, rhenium, ruthenium and osmium, or alloys or mixtures thereof. More preferably the noble metal is gold, platinum, or alloys or mixtures thereof, but is most preferably gold.

The noble metal layer preferably comprises at least a top and bottom surface, and suitably the biological material is located on one of the top and bottom surfaces.

Preferably the biological material is immobilised on the noble metal layer. The biological material is preferably present as a layer on the noble metal layer. The biological material layer is preferably a self-assembled layer.

Suitably the biological material comprises a plurality of sulphur-containing functional groups, preferably sulphydryl (—SH) groups.

Suitably biological material is a protein. Suitably the protein is selected from an enzyme, antibody, receptor, antibody fragment, or binding protein, but is most preferably an enzyme.

Preferably the enzyme is a nitroreductase.

Suitably the nitroreductase is encoded by the nfnB gene (SEQ ID1) in Escherichia coli or the pnrA gene (SEQ ID2) in Pseudomonas putida. (the native enzymes are referred to hereinafter as “nfnB” and “pnrA” respectively). Suitably the nitroreductase is encoded by a nucleic acid sequence substantially as set out in SEQ ID1 or SEQ ID2.

The biological material may be covered by a fluid permeable cover layer, preferably in the form of membrane. The cover layer may comprise a polycarbonate or polyacrylate material and is preferably between 1 and 100 μm in thickness, more preferably between 5 and 50 μm, still more preferably between 8 μm and 30 μm and most preferably between 10 and 20 μm.

The noble metal layer is preferably mounted on an insulating substrate. The insulating substrate may be selected from glass, quartz, silicon, insulating polymers, plastics, and mixtures thereof.

The noble metal layer may be connected on a surface not comprising the biological material, to one or more layers of conductive, semi-conductive or insulating material. Conductive and semi-conductive materials include metals, alloys, carbon paste, graphite and conducting polymers such as polpyrrole, polyaniline, polythiophene, polypyrimidine and the like for example. The one or more further layers may be located between the noble metal layer and the insulating substrate, when present.

In particularly preferred examples the sensing device comprises a gold layer on which is self-assembled a layer of nitroreductase enzyme which has been modified to include a plurality of cysteine residues at a location on the enzyme which does not substantially interfere with the activity of the enzyme.

The nitroreductase may comprise substantially the expression product of the nucleic acid sequence shown in SEQ ID3, which comprises the nucleotide sequence between the T7 promoter and the T7 terminator of the pET-28a(+) plasmid containing the Escherichia coli K12 nfnB gene, in which 6 cysteine residues (a “Cys₆” tag) have been inserted at the N-terminal end.

Another preferred nitroreductase comprises substantially the expression product of the nucleic acid sequence shown in SEQ ID5, which is the nucleic acid sequence between the T7 promoter and the T7 terminator on the plasmid pET-28a(+) containing the Pseudomonas putida JLR11 pnrA gene in which a Cys₆ tag has been inserted at the N-terminal end.

The amino acid translation products of SEQ ID3 and SEQ ID5 are given in SEQ ID4 and SEQ ID6 respectively. Suitably the modified nitroreductase comprises a polypeptide sequence substantially as set out in SEQ ID4 or SEQ IDG.

The nitroreductase may be operably associated with an electron mediator, such as a ferrocene, a phthalocyanate or the like, foe example.

According to a second aspect of the invention there is provided a sensing system comprising a sensing device of the first aspect of the invention, mounted in an electrochemical cell.

The electrochemical cell preferably comprises, in addition to the sensing device, a reference electrode. More preferably the electrochemical cell comprises both a reference electrode and a counter-electrode.

The reference electrode may comprise a Calomel electrode (Standard Calomel Electrode (SCE)), Hg/Hg₂Cl₂ electrode and/or Ag/AgCl electrode, or any combination thereof. Ag/AgCl electrodes are preferred as they may be manufactured in various forms, for example discs, wires, rods, layers etc.

The electrochemical cell may comprise a housing constructed from glass, polystyrene or the like, for example.

The sensing system may be operably connected to a measuring instrument, such as a voltammeter, amperometer, cyclic voltammeter, or the like, for example.

According to a third aspect of the invention there is provided a method of detecting nitro group-containing compounds, the method comprising the steps of:
(a) providing a sensing device of the first aspect of the invention and a reference electrode;
(b) applying a potential between the electrodes;
(c) measuring the current;
(d) contacting the sensing device with sample of substrate material to be tested; and
(e) measuring the current change.

The method may comprise a further step (f) of subtracting the current change measured with a blank electrode from the value obtained in step (e). The blank electrode may be the sensing device of the first aspect of the invention which either does not contain the biological material, or contains inactivated biological material.

Depending on the physical type of the sensing device of the first aspect of the present invention, there may be a step between steps (a) and (b) of placing the sensing device in a measuring solution. In this case step (d) may comprise adding a sample of the material to be tested, to the measuring solution.

According to a fourth aspect of the present invention there is provided a protein comprising a nitroreductase enzyme which has been modified to comprise a plurality of cysteine residues incorporated into its structure.

According to a fifth aspect of the present invention there is provided an isolated nucleic acid sequence comprising a nitroreductase gene modified by the addition of a plurality of codons for cysteine residues.

Suitably the nitroreductase gene is selected from the Escherichia coli K12 nfnB gene and the Pseudomonas putida JLR11 prnA gene. The nucleic acid sequences for the nfnB and pnrA genes are given as SEQ ID1 and SEQ ID2 respectively and preferably the nitroreductase gene is encoded by a nucleic acid sequence substantially as set out in SEQ ID1 and SEQ ID2. Suitably the cysteine codons are incorporated at or in the region of the 3′ end of the nucleic acid.

According to a sixth aspect of the present invention there is provided a nucleic acid construct comprising:
(a) a promotor for the expression of a nitroreductase gene;
(b) a plurality of codons for Cys residues; and
(c) a nucleotide sequence of a nitroreductase gene; Suitably the nitroreductase promoter is the T7 promoter from pET-28a(+).

Suitably the construct comprises the pET-28a(+) plasmid.

Preferably the construct is a vector substantially comprising the nucleic acid sequences shown in SEQ ID3 or SEQ ID5, the reverse complement of the said sequences, the complement of the said sequences, the reverse of the said sequences, or sequences having at least 60% sequence identity with the nucleic acid sequences of any one of the aforementioned sequences.

By use of the term “at least 60% identity” it is therefore understood that the invention encompasses more than use of the specific exemplary nucleotide sequences. Modifications to the sequence such as deletions, insertions, or substitutions in the sequence which produce either:

a) “silent” changes which do not substantially affect the functional properties of the protein molecule. For example, alterations in the nucleotide sequence which reflect the degeneracy of the genetic code or which result in the production of a chemically equivalent amino acid at a give site are contemplated, or:
b) promote improvements in activity or modifications in substrate specificity are also contemplated.

A modification of the nucleotide sequence with an identity greater than 80%, preferably more than 85%, more preferably more than 90% and most preferably more than 95% of SEQ ID 1 or 2 is envisaged.

It has been surprisingly found that the incorporation of cysteine resides in nitroreductases enables efficient incorporation of the nitroreductase onto a noble metal electrode which effects sensitivity of nitrocompound detection down to the picomolar concentration range. Most known nitrocompound detection system enable detection down to the nanomolar range only, and it is believed the conjugation of the nitroreductase and noble metal electrode via cysteine linkages enables optimal orientation of the enzyme on the electrode, leading to enhanced sensitivity. The immobilisation of nitroreductase onto noble metal electrodes via introduced cysteine residues, on the enzyme, is also relatively cheap and uncomplicated. The resultant sensing devices are able to be reused many times and can be used in situ, or in site to detect buried explosives in ground, or in samples taken from suspected explosive-containing materials.

### EXAMPLES

For a better understanding of the present invention and to show how embodiments are the same may be put into effect the invention will now be described by way of example only with reference the accompanying drawings in which:

FIG. 1 illustrates a general model of a modified electrode, showing the mediation of electron transfer.

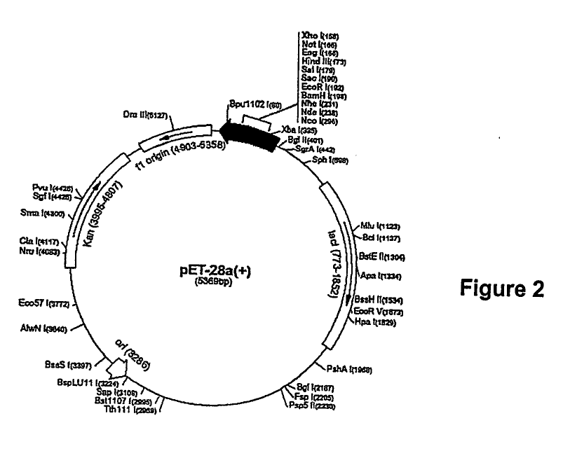

FIG. 2 illustrates the pET-28a(+) plasmid;

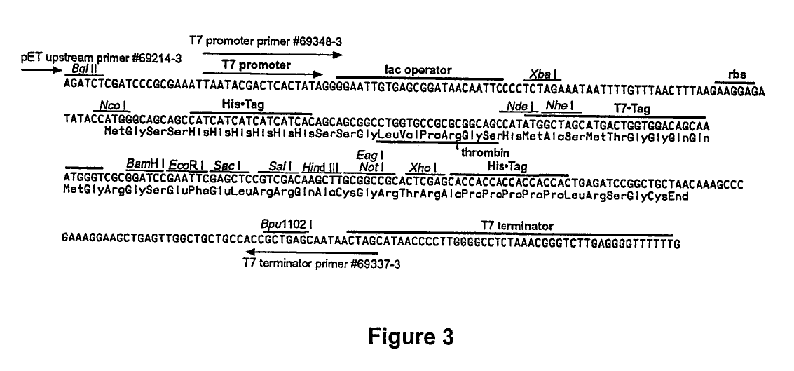

FIG. 3 illustrates a partial nucleotide sequence of the pET-28(a)(+) plasmid from the T7 promoter to the T7 terminator region;

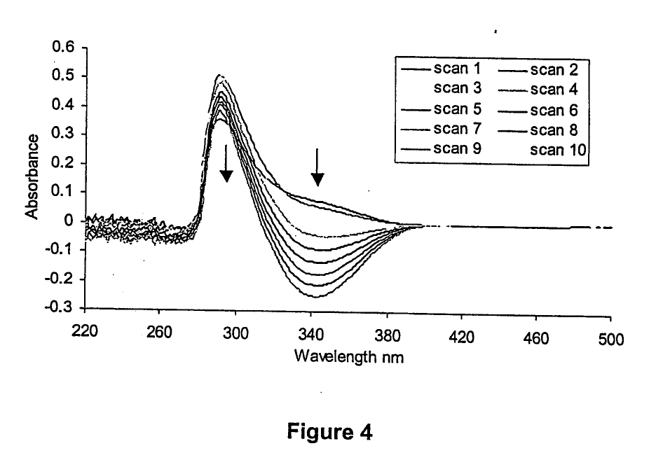

FIG. 4 illustrates a graph showing the influence of a Cys₆ tagged nfnB nitroreductase on a buffer solution containing 4-nitrobenzoate and its UV-viz absorbance spectrum;

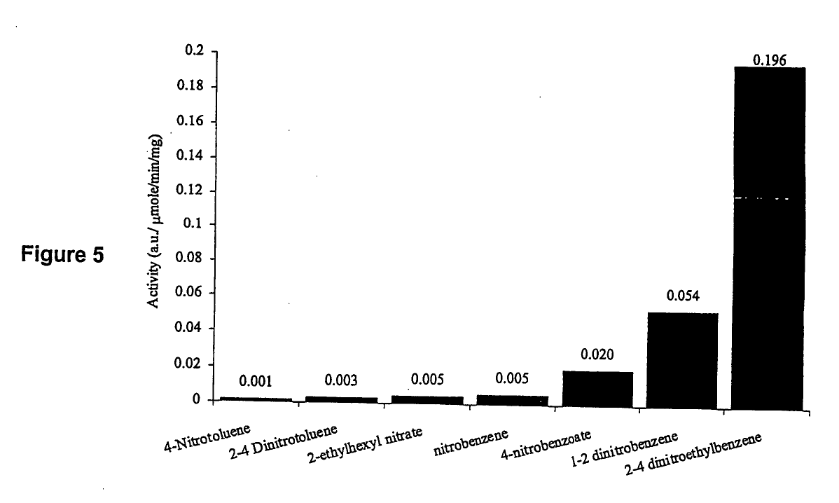

FIG. 5 illustrates a bar chart showing the activity of a Cys₆ tagged nfnB nitroreductase on various substrates;

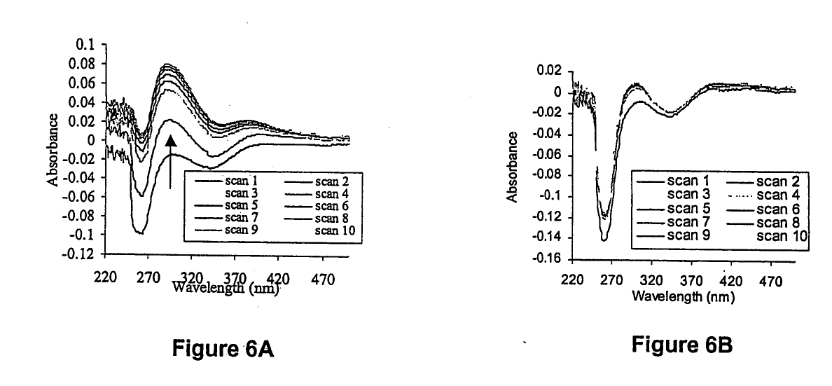

FIGS. 6A and 6B illustrate the activity of desalted and non-desalted forms of a Cys₆ modified nfnB nitroreductase in catalysing breakdown of 2,4-dinitroethylbenzene;

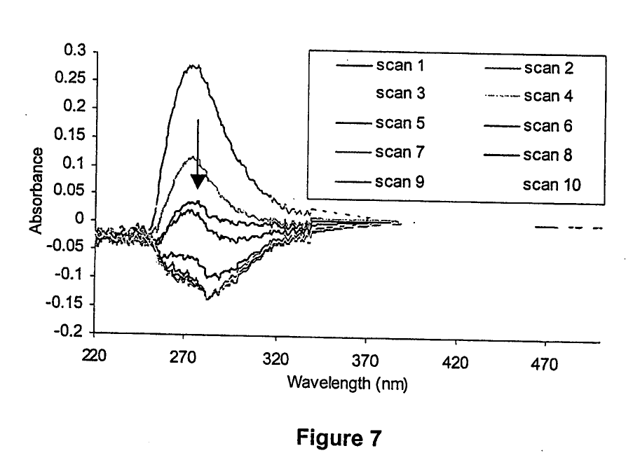

FIG. 7 illustrates activity of a Cys₆ modified nfnB nitroreductase utilising ferrocene dicarboxylic acid as a cofactor;

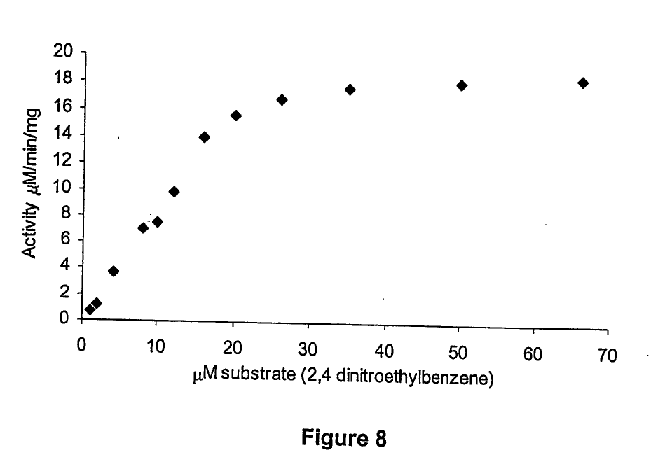

FIG. 8 illustrates a plot of the activity against increasing concentrations of substrates of a Cys₆ modified nfnB nitroreductase;

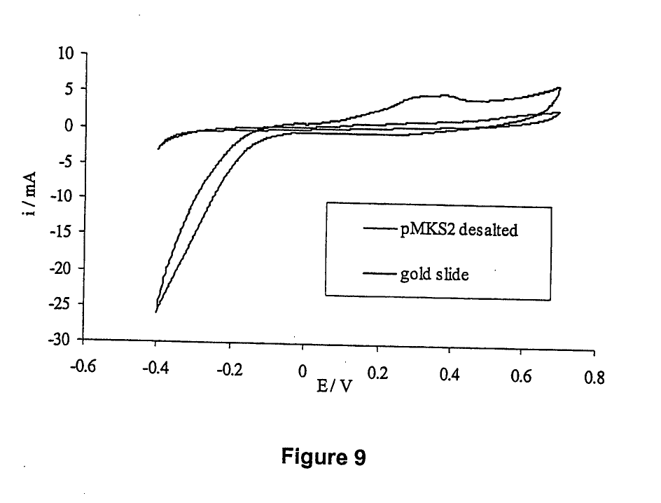

FIG. 9 illustrates a cyclic voltammograms of a Cys₆ modified nfnB nitroreductase immobilised on a golf slide and a control gold slide without attached enzyme;

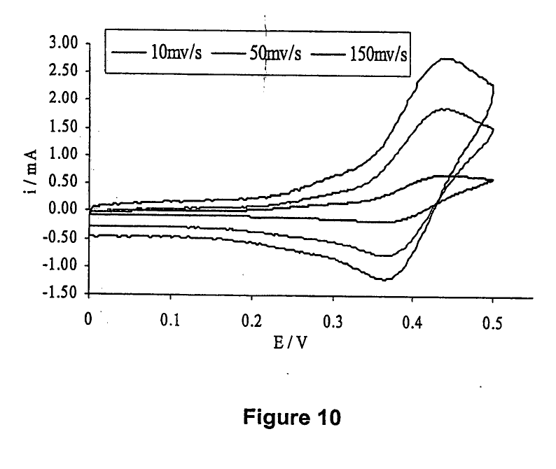

FIG. 10 illustrates cyclic voltammograms of a Cys₆ modified nfnB nitroreductase utilising ferrocene dicarboxlic acid as a cofactor;

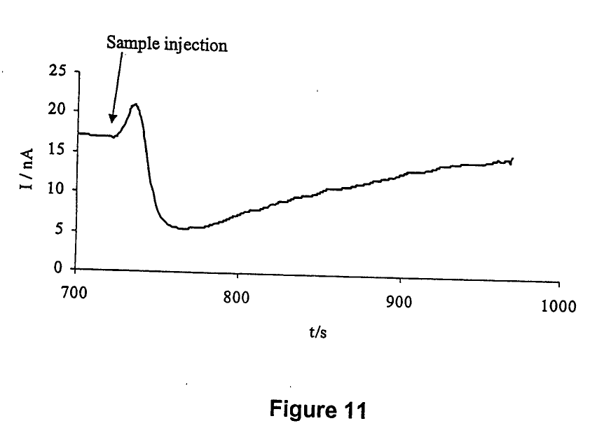

FIG. 11 shows the amperometric response of a biosensor utilising nfnB Cys₆ modified enzyme expressed from a construct at a fixed potential of +100 mV; and

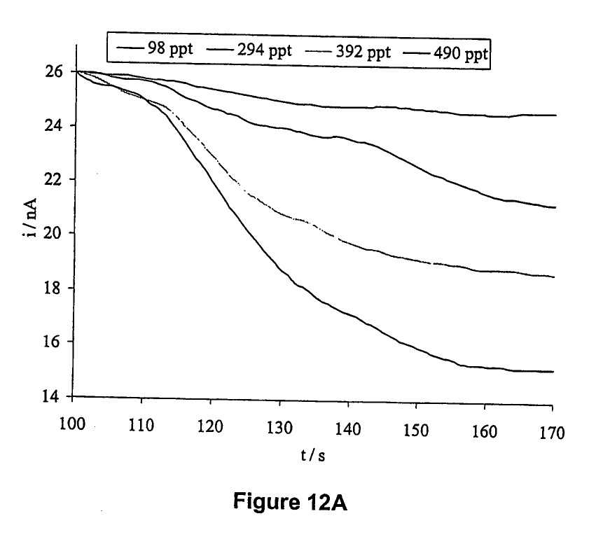

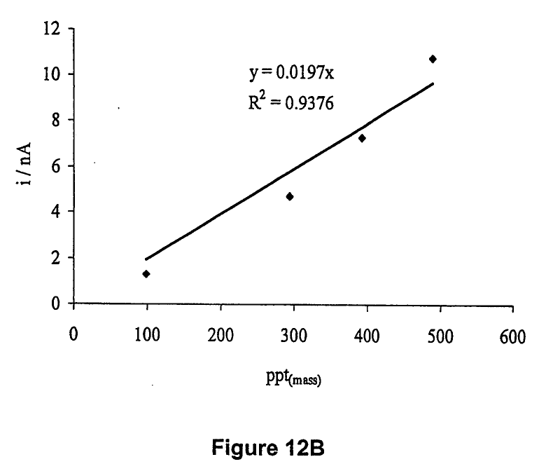

FIGS. 12A and 12B illustrate amperometric measurements of a Cys₆ modified nfnB nitroreductase containing biosensor with 10, 30, 40 and 50 pmoles 2,4 dinitroethylbenzene.

### Example 1

Preparation of a Plasmid Comprising a Modified nfnB gene from Escherichia Coli K12. Plasmids containing nfnB genes from Escherichia coli K12 modified by addition of codons for a Cys₆ tag were prepared in the following manner.

### 1.1 The Procedure for Obtaining the Original DNA Templates

The DNA template was prepared by introducing cells Escherichia coli K12 into a solution of TE buffer pH 7.5 (Tris-Cl, ethylenediaminetetraacetic acid (EDTA)) (100 μl, 1%) in an eppendorf tube. The resulting suspension was mixed thoroughly and boiled for 5 min to break down the cell structure, releasing the DNA into the solution, and was then cooled on ice and centrifuged for 2 min. The centrifuge was set to operate at 14000 rpm unless otherwise stated.

### 1.2 Polymerase Chain Reaction (PCR) Protocol for the Nitroreductase DNA

The PCR protocol uses a standard commercial kit (ProofStart™, Qiagen, UK) according to the manufacturer's instructions.

Template DNA (Escherichia coli K12) (1 μl) was amplified by PCR using the standard procedure with primers conforming to SEQ ID7 (5 μl), and to SEQ ID8 (5 μl)

The PCR system was programmed to run in the following manner. The system was held at 95° C. for 5 min in order to activate the DNA polymerase. The subsequent temperature cycle consisted of 94° C. for 30 s to separate the DNA strands, 62° C. for 1 min for annealing with the primers, 74° C. for 2 min for replicating the double stranded DNA. This sequence was repeated for 35 cycles, after which the temperature was held at 74° C. enabling any uncompleted double strands to complete. An aliquot of the solution was then run on a 1% agarose gel, in 1× Tris-Borate-EDTA (TBE) buffer pH 8.2 containing ethidium bromide. The ethidium bromide acts as a stain enabling the molecular weight and purity of the DNA to be determined by viewing against a sample containing DNA fragments of known molecular size run in the same gel. The remaining DNA from the PCR was purified to remove the primers, nucleotides, polymerase, and salts, in preparation for other enzymatic reactions as follows.

### 1.4 Cloning of the Nitroreductase Gene

Ligation is the incorporation of the DNA into a plasmid. A good efficiency of ligation of foreign DNA into a plasmid can be achieved if both the plasmid and the insert DNA are cut with two different restriction enzymes, which leave single-stranded, cohesive ends. The DNA is thus ligated in only one predetermined direction.

The PCR product (5 μl) was mixed with the appropriate restriction enzymes (2 μl of each), purified water (2 μl), and 10× appropriate restriction buffer solution (1 μl). A separate solution was made up of the expression plasmid pET-28a(+) (5 μl) (Novagen, UK; FIGS. 2 and 3). The region between the T7 promoter and T7 terminator is shown in FIG. 3. This was mixed with the same two restriction enzymes (1 μl of each) plus a third, EcoRI (1 μl), purified water (1 μl), and 10× buffer solution (1 μl): the EcoRI digestion ensures that the pET28a(+) plasmid does not religate without an insert. Each solution was incubated at 370 for 1 hour to allow the restriction digestion to occur, after which each was cleaned up according to section 1.3. Both were dried under vacuum and redissolved in purified water (4 μl).

The two solutions were then mixed and DNA ligase (1 μl), and ligase 10× buffer solution (1 μl), were added. This solution was then maintained overnight at 16° C. for the ligation process. To check that the ligation of the PCR product between the T7 promoter and T7 terminator of pET-28(a)(+) was successful an aliquot of the ligation mix was digested with the appropriate restriction enzymes (1 μl each) as described above and subjected to agarose gel electrophoresis.

The remaining ligation mixture was mixed with cells of competent E. coli DH5a (200 μl), an efficient strain of E. coli for plasmid maintenance. In order to transform the recombinant plasmid into the competent cells, the mixture was left on ice for 30 min, was then heated to 42° C. for exactly 50 sec and then returned to ice for 2 min. The resulting culture was added to Luria-Bertani (LB) medium (500 μl), incubated at 37° C. for 45 min, and then applied to the Petri dishes.

### 1.5 Growing the Colonies

The solid medium consisted of LB agar containing the antibiotic kanamycin (50 μg/ml). This medium will only allow bacteria carrying the recombinant plasmid to grow, as a kanamycin resistance gene is an integral part of pET28a(+). The transformed E. coli culture was spread onto the plates in a range of different concentrations. Single colonies, which grew were then transferred to liquid LB medium (5 ml) containing kanamycin (50 μg/ml) and grown overnight at 37° C.

The recombinant plasmid was isolated from the overnight cultures after separating the cells as a pellet following centrifugation. A standard QIAprep Spin Miniprep Kit (Qiagen, UK) was used to purify the plasmid according to the manufacturer's instructions.

### 1.7 PCR Protocol for the Incorporation of the Cys₆ Sequences.

Using purified pCDG1 as template DNA, the protocol for PCR was repeated using designed primers conforming to SEQ ID9 and to SEQ ID8: these contain six adjacent codons for Cys at the 3′ end of the nitroreductase such that when the gene is expressed an amino acid sequence of Cys₆ is added to the N-terminus of the protein, between the His₆-tag determined by pET28a(+) and the start codon of the nitroreductase. The same protocols as described above for cloning the modified gene, transforming it into E. coli DH5α, and purifying it were used. The resultant plasmid was named pMKS2.

### Example 2

Expression of the Cys₆-modified nitroreductase enzyme prepared in section 1.7, coded by the modified nfnB gene located in plasmid pET-28a(+) prepared in Example 1.

### 2.1 Expression of the Enzymes

Using the protocol of Section 1.4, the plasmids prepared in Example 1 (2 μl) were transformed into competent cells of the Rossetta strain of E. coli (200 μl), which is an efficient bacterium for the expression of heterologous genes.

The bacteria containing the plasmids were grown overnight at 37° C. in 500 ml of LB plus added kanamycin (50 μg/ml), until an optical density (O.D._{600 nm}) of 0.6 was achieved. Expression of the cloned genes was induced by addition of isopropyl-beta-D-thiogalactopyranoside (IPTG) (2 ml, 0.1 M: 0.4 mM^{(final)}), and grown for a further four hours at 37° C.

The cells were then harvested by centrifuging (8000 rpm for 10 min) and the resulting pellets were placed on ice and resuspended in imidazole solution (10 mM, 10 ml) consisting of phosphate buffer (pH 7.4, 6.25 ml, 0.1 M), and imidazole (2 M, 0.25 ml), made up to 50 ml with distilled water. The resulting suspensions were then sonicated four times for 30 s, to break open the cells, whilst avoiding overheating the solution. The solutions were then centrifuged (35000 rpm, 5° C., for 45 min). The resulting solution contains the nitroreductase (NTR) and the pellet contains the cell debris. The solutions were then run on a sodium dodecyl sulphate-polyacrylamide gel electrophoresis to check that the protein was overproduced and that its molecular weight was as expected.

### 2.2 The Enzyme Purification Protocol

The engineered proteins carry a His₆ tag at their N-termini, making it easier to purify the protein by eluting the solution through a nickel-agarose column, where the histidine residues bind to Ni²⁺ embedded in the resin. His-tagged target proteins are thus selectively retained on the column of nickel agarose, and can be eluted (competitively removed) with imidazole, which competes for Ni binding sites, displacing the protein.

For elution from the column, imidazole solutions (8 ml) were prepared with increasing concentrations from 50 mM to 1.0 M in sterile filtered distilled water.

The extract of the cells was added to the column dissolved in phosphate buffer containing 10 mM imidazole. Elution was carried out according to the maker's instructions (Amersham Biosciences U.K.) using imidazole concentrations increasing in stepped amounts. Each eluate was collected in 1 ml samples (5 for each concentration) to avoid dilution. Finally, the column was washed with the remaining binding buffer (4 ml) and stored below 5° C. ready for reuse. The second ml of each elute was run on a SDS-PAGE gel along with the sample flow-through and an induced unpurified sample.

2.3 The Removal of Imidazole from the Protein

The imidazole from the elution stages remains in the enzyme solution. It was removed, as stated below, and the nitroreductase (NTR) resuspended in Tris buffer pH 7.2 ready for use on the electrode surface. A PD-10 desalting column (Amersham Biosciences U.K.) which is a gravity-operated polypropylene column containing 8.5 ml of Sephadex™ G-25 Medium, with a bed height of 5 cm, used for desalting and buffer exchange and was used according to the manufacturer's instructions.

### Example 3

Characterisation of Enzyme Activity. The activity of the modified nitroreductase enzyme prepared in Example 1 was preferred as follows:

### 3.1 Nitroreductase Assay

A UV assay and associated spectra were carried out on the NTR obtained at the end of Section 2.3 in solution (≅1.0 μM^{(Final)}, 10 μl), in a cuvette with buffer tris-HCl pH 7.4 (50 mM, 500 μl), NADPH (1 mM, 100 μl), nitroaromatic compound (substrate, various conc.), and flavin mononucleotide (FMN) (1 mM, 5 μl), made up to 1 ml with distilled water at 25° C.

The spectra were collected at a scan rate of 500 nm/min between 220-500 nm resulting in 1 min scans, and the assays were run at 340 nm for 2 min each, using an Uvikon 943 double beam spectrophotometer. The parameters of the spectra and assays are as noted above unless otherwise stated. All spectral measurements were carried out against a blank consisting of the assay solution detailed above, but lacking the nitroreductase enzyme, and all assay measurements were carried out against a blank consisting of the assay solution lacking the substrate, unless otherwise stated.

### Example 4

Preparation of an enzyme biosensor utilising the modified nitroreductase of Example 2.

### 4.1 Preparation of the Gold Sheet for UV-vis

Gold was used to prove the concept that the modified enzymes can be immobilised via the thiol groups to gold substrate and remain active after the immobilisation. Gold sheets were cleaned in a 50:50 mixture of concentrated sulphuric and nitric acid overnight. Desalted and non-desalted Cys-tagged NTR enzymes were each/separately adsorbed onto a gold slide, 3 mm by 5 mm. The gold sheet was left for 24 hours in the nitroreductase solution, and then immersed in buffer (pH 7) to remove any residual proteins.

### 4.2 Electrochemical Procedure

Electrochemical measurements were performed using an Autolab PGstat3. The analysis was carried out with a three-electrode cell, using a Saturated Calomel reference electrode (SCE) and a platinum mesh counter electrode. All glassware was cleaned using a 50:50 mixture of concentrated H₂SO₄: HNO₃ followed by rinsing in purified water, cleaning in a steam bath, and drying in the oven. The working electrode was a gold slide with a self-assembled layer of the appropriate enzyme.

The cell contained sodium phosphate buffer pH 7.1 (20 ml, 0.1 M), mixed with the co-factor dicarboxylic ferrocene (50 □M). Additions of the substrate were made by pipetting the desired quantities and concentrations in through the top of the cell.

### 4.3 Preparation of Gold-Coated Glass Slides for Electrochemical Measurements

The gold-coated glass slides obtained from Gold Arrandee™/Au(111) uses the borosilicate glass (AF45) base material, which is 1.1+/−0.1 mm in thickness with the size of the glass slide being 11×11+/−0.2 mm. The special glass substrate is well suited for the flame annealing procedure which is used to obtain Au(111) terraces. A thin (2.5+/−1.5 nm) adhesive layer of chromium is applied to the glass surface. This layer guarantees optimum adhesion of the gold layer to the glass. On top of this thin Chromium layer a final gold layer is applied which is 250+/−50 nm thick.

Prior to use, the gold-coated slides were flame-annealed in a Bunsen burner until they attained red heat several times. After cooling in air for a short period of time, the slide was quenched in ultrapure water. The slides were then dipped into an enzyme-containing solution for 24 hrs at 5° C. to assemble the layer of the enzyme. Each slide was then washed in tris-buffer prior to transfer to an electrochemical cell.

### 5.1 Assay Results

The influence of the nitroreductase on a buffer solution containing 4-nitrobenzoate (625 μM) was assessed using UV-vis; the nitroreductase (10 μl) was placed in the cuvette prior to run 2. The results are shown in FIG. 4.

The scans show a reduction in the intensity of absorbance at 340 nm on the second scan, followed by reduction of the absorbance in subsequent scans, down to −0.29 absorbance units (a.u.). The peak at 300 nm (4-nitrobenzoate) increased in intensity on the second scan due to introduction and corresponding absorbance of nitroreductase, then decreased on subsequent scans. The use of increasing amounts of nitroreductase afforded a corresponding increase in NADPH conversion, indicating that the nitroreductase is responsible for the oxidation of NADPH.

As substrates, 4-nitrotoluene, 2,4-dinitrotoluene, 2-ethylhexyl nitrate and nitrobenzene were good substrates whereas 4-nitrobenzoate, 1,2-dinitrobenzene and 2,4-dinitroethylbenzene were excellent substrates (FIG. 5).

Both desalted and non-desalted forms of the protein NfnB-cys1 nitroreductase with cysteine tags as prepared hereinabove were adsorbed onto gold slides and scans of the activity towards 2,4-dinitroethylbenzene (DNEB) (620 μmoles) were assessed. The results are shown in FIGS. 6A and 6B which clearly illustrate the effective catalysis by the desalted enzyme (FIG. 6A).

NADPH is not an efficient co-factor in electrochemical cells because the oxidised form NADP⁺ produced in the enzyme-catalysed reaction cannot be stoichiometrically reduced to a biologically active form of NADPH electrochemically. For this reason ferrocene dicarboxylic acid (1 μm) was used to eliminate this problem, as it exhibits good electrochemical reversibility. The results are shown in FIG. 7, using 300 μm DNEB with nitroreductase.

The oxidation of the dicarboxylic ferrocene can be seen in the reduced absorbance with time at 280 nm indicating that the ferrocene derivative is being oxidised via the enzymatic reaction, hence, can be utilised as the nitroreductase co-factor.

### 5.2 Specific Activity of Nitroreductase

The specific activity of nitroreductase was assessed by calculating the rates at different concentrations of substrate in association with the different nitroreductase concentration. The preferred substrate used for both the His-tagged nitroreductase from E. coli (NfnB-his1) and the further modified Cys-tagged enzyme (NfnB-cys1) was 2,4-dinitroethylbenzene (31 μM). The protein concentration was calculated by placing a micro protein-PR™ reagent (1 ml) in 3 cuvettes, a blank, a standard (500 mg/l), and a sample of nitroreductase. UV measurements were taken at 600 nm in accordance with the procedure. The unpurified protein was compared against the purified protein. Four runs were performed for each volume of 5, 10, 15, and 20 μl of protein.

The activity for the enzyme NfnB-his1 before purification was 0.22 □moles/min/mg which rose to 4.65 □moles/min/mg following purification. Hence, the purification achieved an approximate average of 20-fold increase in activity, with the enzyme making up only 7.6% of the unpurified solution. The relevant specific activities are tabulated in Table 1.

TABLE 1
The concentration and specific activity for the
unpurifed and purified proteins NfnB-his1 and NfnB-cys1
with 2,4-dinitroethylbenzene.

| | Average | Average |
|---|---|---|
| | total | specific |
| | protein | activity |
| Protein | (□g/ml) | (□moles/min/mg) |
| NfnB-his1 | | |
| Unpurifed | 5.23 | 0.22 |
| Purified | 0.40 | 4.64 |
| NfnB-cys1 | | |
| Unpurifed | 4.83 | 0.18 |
| Purified | 0.18 | 2.92 |

The protein with the Cys₆ tags (NfnB-cys1) showed that the insertion of the Cys residues reduced the activity by an average of ≈37%. After the removal of imidazole from the protein solutions containing the Cys tags, the proteins retained approximately 86% of its original activity.

### 5.3 Kₘ and Vₘₐₓ of the Nitroreductase

In order to calculate the Kₘ and Vₘₐₓ values, a plot was constructed of activity in □□ min/mg against increasing concentration of substrates as shown in FIG. 8.

The resulting data was used to calculate the Kₘ and Vₘₐₓ values from Direct Linear method via an enzyme kinetics theory and practice software package (Enzpack). The resulting data was averaged and obtained with a 68% confidence level, as shown in Table 2 below.

TABLE 2
Resulting K_{M} and Vₘₐₓ values for the
E coli nitroreductase

| | Kₘ | Vₘₐₓ | Vₘₐₓ/Kₘ | |
|---|---|---|---|---|
| Proteins | (μmol) | (μmol/min/mg) | ratio | Substrate |
| NfnB-his1 | 27 | 27 | 0.99 | 2,4- |
| | | | | dinitroethylbenzene |
| NfnB-cys1 | 33 | 27 | 0.84 | 2,4- |
| | | | | dinitroethylbenzene |

The values of Kₘ and Vₘₐₓ are specific to each enzyme however, the ratio Vₘₐₓ/Kₘ can be used to compare the enzyme efficiency, as shown in Table 2. The efficiency of the enzyme is ultimately limited by the rate of diffusion of the substrate to the enzyme and by the chemical events that occur in the active site of the enzyme.

The number of moles of substrate converted to product per unit of time, known as the turnover number (K_{cat}), also allows comparisons between the enzymes. The turnover number can be calculated from the following equation:

Vₘₐₓ=K_{cat}[E_{Tot}]

The average K_{cat} of substrate for the two modified NfnB was 6.4×10² mol/min/moles of enzyme.

### 5.4 Evaluation of the Biosensor; Voltammetry Results

Cyclic voltammograms were obtained for the unmodified gold electrode and the gold electrode modified with the desalted enzyme solution; a seal was made between the working electrode and the electrolyte solution with o-rings defining a geometric area of 0.6 cm². The modification of the electrodes was achieved by immersing the gold slide, which is now the working electrode, in the enzyme solution (0.1 M phosphate butter, pH 7.1) for 24 hours, then thoroughly rinsing with sodium phosphate buffer. The results are shown in FIG. 9.

The reduction in the oxidation peak (oxidation of gold surface) at +350 mV is caused by the formation of the layer of enzymes blocking the electrode surface. The presence of the layer is also manifested by the large reduction in the current associated with the hydrogen evolution reaction at −400 mV. In order for the reaction (reduction of nitroaromatics) to proceed, a co-factor needs to be introduced into the solution. A voltammogram of the co-factor, dicarboxylic ferrocene (35 μm) in 0.1 M phosphate butter, pH 7.1, was carried out to evaluate an appropriate potential to hold the amperometric sensor at, so that the dicarboxylic ferrocene could be reduced after it has been oxidised during the enzymatic reaction. The results are shown in FIG. 10.

FIG. 11 shows the amperometric response of the biosensor with the NfnB-cys1 enzyme expressed from pMKS2 which was evaluated at a fixed potential of +100 mV in a 0.1 M phosphate buffer (pH 7.1) and the co-factor was dicarboxylic ferrocene (10 mM, 200 □moles). The working electrode was a gold slide modified with NfnB-cys1. and all potential values are quoted against a SCE. Prior to the injection of DNEB (20 μl of 2 □M solution of DNEB resulting in a concentration of 2 nM in the cell), the current was allowed to stabilise for 730 s until a steady state current was reached. After the addition of the DNEB sample, a slight increase in current was observed, thought to be due to convection caused by introducing the substrate.

After the initial rise, the current becomes less positive by approximately 10 nA. This drop was caused by the reduction of the ferrocenium dicarboxylic acid that had been formed as a result of the oxidation of ferrocene dicarboxylic acid by the oxidised form of the nitroreductase. The current then starts to decay to a value close to the initial baseline value thus allowing successive readings to be taken. Further amperometric measurements were carried out with 10, 30, 40, and 50 pmoles of DNEB corresponding to 0.5 nM, 1.5 nM, 2 nM, 2.5 nM respectively. The larger the amount of DNEB that is introduced into the system, the larger is the drop. A linear relationship is found between the magnitude of the current and the concentration of the analyte; thus providing a basis for an amperometric sensor. The results are shown in FIGS. 12A and 12B. No response was obtained when DNEB was not present in the analyte solution.

FIG. 12A shows amperometric data for the four different concentrations of DNEB at a potential of +100 mV in a 0.1 M phosphate buffer (pH 7.1), the working electrode was a gold slide modified with NfnB-cys1. DNEB samples were injected 100 s after applying the potential; the co-factor was ferrocene dicarboxylic (10 mM, 200 μmoles). FIG. 12B shows a plot of the magnitude of the current drop taken 70 after the injection of DNEB from the data in FIG. 12A against the concentration of DNEB. The plot is that of a straight line and shows a linear relationship between the magnitude of the current and the concentration of the analyte.

The lowest concentration examined in this case corresponds to a concentration of DNEB in the parts per trillion range (ppt).

### Example 6

The above protocols and tests from Sections 1.1 to 5.4 were performed utilising the Pseudomonas putida JLR11 prnA gene conforming to SEQ ID2 in a pET-28a(+) expression vector comprising the expression sequence shown in SEQ ID4. The results again indicated that excellent sensitivity, in the picomolar range, was exhibited by the resultant biosensor.

### CONCLUSIONS

The results illustrate that the introduction of the cysteine tags at the N-terminus does not reduce the activity in a way that (detrimentally affects/prevents) amperometric measurements, and that the tags were successful in the immobilisation of the enzyme to a gold surface, without the loss of activity. Evidence for the assembly of the nitroreductases on the gold surface was obtained by FTIR, UV-vis spectroscopy, and cyclic voltammetry.

The nitroreductase was shown to be active with a range of nitroaromatics and a nitro ester, namely 2-ethylhexyl nitrate, and afforded different rates of reaction for each substrate. The optimum pH (pH 7.1) and temperature (<40° C.) for the enzyme were established along with K_{M}, Vₘₐₓ, and turnover numbers (K_{cat}).

The response of the amperometric sensor was in the nanoamp range and detection was unexpectedly down in the parts per trillion region. The drop in current (and the rate of drop) was found to be proportional to the concentration of nitroaromatics in solution, and the system showed evidence of recovery after each sample, allowing successive samples to be taken. In addition, the enzyme remained active when kept in the fridge for a period of two weeks.

1. A sensing device comprising an electrode comprising a noble metal layer, on which layer is located a biological material having nitroreductase activity.

2. A sensing device as claimed in claim 1 wherein the noble metal layer comprises a noble metal selected from the group consisting of gold, silver, platinum, palladium, iridium, rhenium, ruthenium and osmium, or alloys or mixtures thereof.

3. A sensing device as claimed in claim 1 wherein the biological material is immobilised on the noble metal layer.

4. A sensing device as claimed in claim 1 wherein the biological material is preferably present as a layer on the noble metal layer.

5. A sensing device as claimed in claim 1 wherein the biological material comprises a plurality of sulphur-containing functional groups.

6. A sensing device as claimed in claim 1 wherein the biological material is a protein.

7. A sensing device as claimed in claim 6 wherein the biological material is a nitroreductase enzyme.

8. A sensing device as claimed in claim 7 wherein the nitroreductase is encoded by a nucleic acid sequence substantially as set out in SEQ ID1 or SEQ ID2.

9. A sensing device as claimed in claim 1 wherein the biological material is covered by a fluid permeable cover layer.

10. A sensing device as claimed in claim 9 wherein the cover layer comprises a polycarbonate or polyacrylate material.

11. A sensing device as claimed in claim 1 wherein the noble metal layer is mounted on an insulating substrate.

12. A sensing device as claimed in claim 1 wherein the noble metal layer is connected on a surface not comprising the biological material, to one or more layers of conductive, semi-conductive or insulating material.

13. A sensing device as claimed in claim 1 comprising a gold layer on which is self-assembled a layer of nitroreductase enzyme which has been modified to include a plurality of cysteine residues at a location on the enzyme which does not substantially interfere with the activity of the enzyme.

14. A sensing device as claimed in claim 13 wherein the nitroreductase enzyme comprises substantially the expression product of the nucleic acid sequence shown in SEQ ID3 or SEQ ID5.

15. A sensing device as claimed in claim 14 wherein the nitroreductase enzyme comprises a polypeptide sequence substantially as set out in SEQ ID4 or SEQ ID6.

16. A sensing device as claimed in claim 13 wherein the nitroreductase is operably associated with an electron mediator.

17. A sensing system comprising a sensing device of claim 1, mounted in an electrochemical cell.

18. A sensing system as claimed in claim 17 wherein the electrochemical cell comprises, in addition to the sensing device, a reference electrode.

19. A sensing system as claimed in claim 18 wherein the electrochemical cell further comprises a counter-electrode.

20. A method of detecting nitro group containing compounds, the method comprising the steps of:
(a) providing a sensing device of claim 1 and a reference electrode;
(b) applying a potential between the electrodes; (c) measuring the current;
(d) contacting the sensing device with a sample of substrate material to be tested; and
(e) measuring the current change.

21. A method as claimed in claim 20 further comprising a step (f) of subtracting the current change measured with a blank electrode from the value obtained in step (e).

22. A method as claimed in claim 20 further comprising a step between steps (a) and (b) of placing the sensing device in a measuring solution.

23. A protein comprising a nitroreductase enzyme which has been modified to comprise a plurality of cysteine residues incorporated into its structure, which cysteine residues are not present in the native enzyme.

24. An isolated nucleic acid sequence comprising a nitroreductase gene modified by the addition of a plurality of codons for cysteine residues.

25. A nitroreductase enzyme as claimed in claim 23 encoded by a nucleic acid sequence essentially as set out in SEQ ID1 or SEQ ID2.

26. A nucleic acid construct comprising:
(a) a promoter for the expression of a nitroreductase gene;
(b) a plurality of codons for cysteine residues; and
(c) a nucleotide sequence of a nitroreductase gene.

27. A nucleic acid construct as claimed in claim 26 comprising the nucleic acid sequence set out in SEQ ID3 or SEQ IDS, the reverse complement of said sequences, the complement of said sequences, the reverse of said sequences or sequences having at least 6096 sequence identity with the nucleic acid sequences of any one of said sequences.


SEQUENCE LISTING (9 sequences)

SEQ ID NO: 1 - DNA, 654 bp, Escherichia coli
  K12
atggatatcatttctgtcgccttaaagcgtcattccactaaggcatttgatgccagcaaaaaacttaccccggaacaggccgagcagatcaaaacgctactgcaatacagcccatccagcaccaactcccagccgtggcattttattgttgccagcacggaagaaggtaaagcgcgtgttgccaaatccgctgccggtaattacgtgttcaacgagcgtaaaatgcttgatgcctcgcacgtcgtggtgttctgtgcaaaaaccgcgatggacgatgtctggctgaagctggttgttgaccaggaagatgccgatggccgctttgccacgccggaagcgaaagccgcgaacgataaaggtcgcaagttcttcgctgatatgcaccgtaaagatctgcatgatgatgcagagtggatggcaaaacaggtttatctcaacgtcggtaacttcctgctcggcgtggcggctctgggtctggacgcggtacccatcgaaggttttgacgccgccatcctcgatgcagaatttggtctgaaagagaaaggctacaccagtctggtggttgttccggtaggtcatcacagcgttgaagattttaacgctacgctgccgaaatctcgtctgccgcaaaacatcaccttaaccgaagtgtaa

SEQ ID NO: 2 - DNA, 826 bp, Pseudomanas putida
  JLR11
atgagccttcaagacgaagcactcaaagcctggcaagcccgttatggcgagccagctaacttacctgctgccgacaccgtgatcgcgcagatgttgcagcatcgatcagtacgtgcctacagcgatcttcctgtggatgagcagatgctgagctgggcgatcgcggcggcccagtcagcctcgacttcctcgaacctgcaagcttggagcgtgctcgccgtgcgggatcgcgagcgtctcgcgaggcttgcccgactgtccggtaaccagcgccatgtcgagcaggcaccgctgttcctggtctggctcgtggactggtcacgcctacgccgactagccagaacccttcaggcaccgactgcaggtatcgactatttagaaagctacaccgtcggtgttgtagatgcagctctggccgctcagaacgccgcactagctttcgaggcccaaggactgggaatcgtttacatcggcggaatgcgcaaccacccggaagcgatgtccgaggagcttggcctgccaaacgacactttcgctgtatttggcatgtgcgtcggtcatcccgatccggcacagcccgccgagatcaagccacgcctggcgcaatcagtggtgcttcaccgtgagcgctatgaggccaccgaggcagaggcggtttcagttgctgcctatgaccgaaggatgagcgacttccaacatcgtcaacaacgcgaaaaccgttcctggtccagccaggccgtggaacgtgtaaaaggagcggattcactgagcggaagacaccgcttgcgagatgcattaaacaccctaggtttcggcctgcgctgag

SEQ ID NO: 3 - DNA, 1066 bp, Escherichia coli
  K12 nfnB in pET-28(a)(+); pMKS2
taatacgactcactataggggaattgtgagcggataacaattcccctctagaaataattttgtttaactttaagaaggagatataccatgggcagcagccatcatcatcatcatcacagcagcggcctggtgccgcgcggcagccatatggctagcatgactggtggacagcaaatgggtcgcggatcctgttgctgttgctgttgcgatatcatttctgtcgccttaaagcgtcattccactaaggcatttgatgccagcaaaaaacttaccccggaacaggccgagcagatcaaaacgctactgcaatacagcccatccagcaccaactcccagccgtggcattttattgttgccagcacggaagaaggtaaagcgcgtgttgccaaatccgctgccggtaattacgtgttcaacgagcgtaaaatgcttgatgcctcgcacgtcgtggtgttctgtgcaaaaaccgcgatggacgatgtctggctgaagctggttgttgaccaggaagatgccgatggccgctttgccacgccggaagcgaaagccgcgaacgataaaggtcgcaagttcttcgctgatatgcaccgtaaagatctgcatgatgatgcagagtggatggcaaaacaggtttatctcaacgtcggtaacttcctgctcggcgtggcggctctgggtctggacgcggtacccatcgaaggttttgacgccgccatcctcgatgcagaatttggtctgaaagagaaaggctacaccagtctggtggttgttccggtaggtcatcacagcgttgaagattttaacgctacgctgccgaaatctcgtctgccgcaaaacatcaccttaaccgaagtgtaattctctcttgccgggcatctgcccggctatttcctctcagattctcctgatttgcataaccctgtttcagcaagcttcgtcatcataggctgctgttgaagcttgcggccgcactcgagcaccaccaccaccaccactgagatccggctgctaacaaagcccgaaaggaagctgagttggctgctgccaccgctgagcaataactagc

SEQ ID NO: 4 - protein, 256 aa, Escherichia coli
  K12 nfnB in pET-28(a)(+); pMKS2
MGSSHHHHHHSSGLVPRGSHMASMTGGQQMGRGSCCCCCCDIISVALKRHSTKAFDASKKLTPEQAEQIKTLLQYSPSSTNSQPWHFIVASTEEGKARVAKSAAGNYVFNERKMLDASHVVVFCAKTAMDDVWLKLVVDQEDADGRFATPEAKAANDKGRKFFADMHRKDLHDDAEWMAKQVYLNVGNFLLGVAALGLDAVPIEGFDAAILDAEFGLKEKGYTSLVVVPVGHHSVEDFNATLPKSRLPQNITLTEV

SEQ ID NO: 5 - DNA, 1221 bp, Pseudomonas putida
  JLR11 prnB in pET-28(a)(+); pKMS6
taatacgactcactataggggaattgtgagcggataacaattcccctctagaaataattttgtttaactttaagaaggagatataccatgggcagcagccatcatcatcatcatcacagcagcggcctggtgccgcgcggcagccatatggctagcatgactggtggacagcaaatgggtcgcggatcctgttgctgttgctgttgcagccttcaagacgaagcactcaaagcctggcaagcccgttatggcgagccagctaacttacctgctgccgacaccgtgatcgcgcagatgttgcagcatcgatcagtacgtgcctacagcgatcttcctgtggatgagcagatgctgagctgggcgatcgcggcggcccagtcagcctcgacttcctcgaacctgcaagcttggagcgtgctcgccgtgcgggatcgcgagcgtctcgcgaggcttgcccgactgtccggtaaccagcgccatgtcgagcaggcaccgctgttcctggtctggctcgtggactggtcacgcctacgccgactagccagaacccttcaggcaccgactgcaggtatcgactatttagaaagctacaccgtcggtgttgtagatgcagctctggccgctcagaacgccgcactagctttcgaggcccaaggactgggaatcgtttacatcggcggaatgcgcaaccacccggaagcgatgtccgaggagcttggcctgccaaacgacactttcgctgtatttggcatgtgcgtcggtcatcccgatccggcacagcccgccgagatcaagccacgcctggcgcaatcagtggtgcttcaccgtgagcgctatgaggccaccgaggcagaggcggtttcagttgctgcctatgaccgaaggatgagcgacttccaacatcgtcaacaacgcgaaaaccgttcctggtccagccaggccgtggaacgtgtaaaaggagcggattcactgagcggaagacaccgcttgcgagatgcattaaacaccctaggtttcggcctgcgctgagatagtgagatatcccatgcctattcccgccgccctgaaccggagcactaatacctggcaactttgcttgagctccgtcgacaagcttgcggccgcactcgagcaccaccaccaccaccactgagatccggctgctaacaaagcccgaaaggaagctgagttggctgctgccaccgctgagcaataactagc

SEQ ID NO: 6 - protein, 313 aa, Pseudomonas putida
  JLR11 prnB in pET-28(a)(+); pKMS6
MGSSHHHHHHSSGLVPRGSHMASMTGGQQMGRGSCCCCCCSLQDEALKAWQARYGEPANLPAADTVIAQMLQHRSVRAYSDLPVDEQMLSWAIAAAQSASTSSNLQAWSVLAVRDRERLARLARLSGNQRHVEQAPLFLVWLVDWSRLRRLARTLQAPTAGIDYLESYTVGVVDAALAAQNAALAFEAQGLGIVYIGGMRNHPEAMSEELGLPNDTFAVFGMCVGHPDPAQPAEIKPRLAQSVVLHRERYEATEAEAVSVAAYDRRMSDFQHRQQRENRSWSSQAVERVKGADSLSGRHRLRDALNTLGFGLR

SEQ ID NO: 7 - DNA, 24 bp, Escherichia coli
ggatccgatatcatttctgtcgcc

SEQ ID NO: 8 - DNA, 27 bp, Escherichia coli
cgtcatcataggctgctgttgaagctt

SEQ ID NO: 9 - DNA, 42 bp, Artificial Sequence
  Description of Artificial Sequence Synthetic primer
ggatcctgttgctgttgctgttgcgatatcatttctgtcgcc